(19) United States
(12) Patent Application Publication
(10) Pub. No.: US 2024/0228340 A1
(43) Pub. Date: Jul. 11, 2024

(54) SYSTEM AND PROCESS FOR MEASURING OF A GAS DISSOLVED IN A LIQUID

(71) Applicant: Searas AS, Bergen (NO)

(72) Inventors: Eldar Lien, Bergen (NO); Vincent Frederic Marie Martinez, Bergen (NO); Morten AGA, Bergen (NO); Jan Vidar Nordstrand, Oslo (NO); Geir Valsvik, Laksevåg (NO)

(21) Appl. No.: 18/558,798

(22) PCT Filed: May 11, 2022
(86) PCT No.: PCT/NO2022/000002

§ 371 Date: Nov. 3, 2023

(30) Foreign Application Priority Data
May 12, 2021 (NO) 20210604

(51) Int. Cl. C02F 1/66; C02F 1/00; C02F 1/68; G01N 33/18; A01K 63/04; C02F 101/16; C02F 103/20

(52) U.S. Cl. CPC C02F 1/66; C02F 1/008; C02F 1/68; G01N 33/18; A01K 63/042; A01K 63/045; C02F 2101/16; C02F 2103/20; C02F 2209/14

(57) ABSTRACT

A system and method to measure the amount of a gas dissolved in a fluid is described. The fluid is transferred to an equilibrator and pH is adjusted so that the equilibrium between the gas and its ions in the fluid is displaced towards more gas, so that the measurement may be carried out when there is proportionally more gas in the fluid.

### FIELD OF THE INVENTION

The present invention relates to a system and a method to measure the amount of a gas dissolved in a fluid. More precisely, the invention relates to a system and method for measuring the amount of a gas dissolved in a fluid, where the pH of the fluid is adjusted before the amount of gas is measured. The invention is particularly suited to measure gases in fluids where the pH-adjustment of the fluid changes the equilibrium of the gas dissolved in the fluid so that the amount of said gas in the fluid increases.

### BACKGROUND OF THE INVENTION

In farming installations for marine organisms, such as fish, ammonium (NH₃) will be generated from faeces and the fishes' exhalation of air. Concentrations of ammonium in such farming fluids are typically in the range of 1-30 ppb (parts per billion). The ammonium formed in the farming fluid is poisonous to the fish and must be removed from the water. It is especially important to remove dissolved ammonium from the water in farming installations where the water is recycled back into the plant, i.e., in so called RAS installations. The removal of ammonium dissolved in the water is carried out in the biofilters of the installation, where ammonium (NH_{3(aq)} is converted to nitrite (NO₂(aq)) and nitrate (NO_{3(aq)}).

The concentration of NH₃ in the fluid is so low that it is practically impossible to measure the concentration.

### Review of Prior Art

Systems exist to continuously measure the concentration of a gas dissolved in a fluid, where the fluid is transported to a closed container, and where the amount of gas is measured during the gas phase above the fluid in the closed container. Such a system is described in U.S. Pat. No. 3,942,792 where the container is arranged in the tank itself where the fluid is collected. However, such a system cannot measure the low amounts of NH₃ dissolved in a fluid.

U.S. Pat. No. 5,882,937 describes a system to regulate the amount of NH₃ in water, where an alkaline or an acid is added to the fluid container itself, by the fluid being brought into contact with an alkaline or acid containing solid phase. Such a solution is impossible to use when measuring the concentration of gases in a farm installation, as the pH adjustment will lead to injury and death of the fish.

### Objective of the Present Invention

The present invention has as an objective to provide an improved system and method for measuring the concentration of a gas dissolved in a fluid.

It is an objective to enable continuous measurement of the concentration of a gas in the fluid, i.e., without having to obtain samples from the fluid for analysis.

In particular, it is an objective of the invention to enable measurement of the amount of NH₃ gas dissolved in fluid, such as a fluid for the farming of marine organisms, like fish. Thus, it will be appropriate to use the system and method of the invention to measure the amount of NH₃ dissolved at any time in the farming installation, but the system and method may also be used to measure other gases dissolved in fluid, and may be used for other fluids, such as tap water, purification plants, etc. Furthermore, it is an objective of the invention that the adjustment of the pH is not performed in the farming fluid in the tank itself, i.e., that the pH adjustment takes place downstream of the farming tank and is transferred to a separate container to measure the amount of gas.

### SUMMARY OF THE INVENTION

The present invention relates in a first aspect to a system to determine the amount of a gas dissolved in a fluid in a container, wherein the system comprising an equilibrator arranged to set an equilibrium between gases in a gas phase and fluid phase, a sensor device to measure the amount of gas in the gas phase, and a container upstream of the equilibrator to regulate the pH of the fluid before it is transferred to the equilibrator.

In a preferred embodiment, the container is arranged to regulate the pH in the fluid and comprises means to add a pH regulating agent to the container.

In preferred embodiments, the pH regulating agent is in the form of a gas, a fluid, or a solid.

In one embodiment, the system comprises a gas transporter arranged to cause circulation of gases from the gas phase to the fluid phase.

In one embodiment, the equilibrator has an outlet with a water lock to regulate the fluid level in the equilibrator.

In one embodiment, the sensor device measures the amount of one or many gasses directly in the gas phase in the equilibrator.

In one embodiment, one or many gases are added to the fluid phase in the equilibrator.

In one embodiment, the said one or many gases is air or oxygen.

In one embodiment, the gas transporter transports gases in a closed circuit from the gas phase to the fluid phase in the equilibrator.

In one embodiment, the gas transporter comprises a pump and a pipeline for transport of gases from the gas phase to the fluid phase in the equilibrator.

In one embodiment, the system comprises a closed loop and that gases from the gas phase are transported by a gas transporter to the fluid phase in the equilibrator via this loop, and that a sensor device is arranged in the loop, and which measures the amount of one or many gases in the gas phase.

In one embodiment, the gas from the gas phase is directed in a closed loop via a sensor device to measure the amount of a specific gas.

In one embodiment, the gas supply unit is a hose equipped with an air pump to pick up gas from the gas phase and add it to the fluid phase in the equilibrator.

In one embodiment, the gas transporter is an ejector.

In one embodiment, the fluid is fed via pump and pipelines to the top of the equilibrator and the ejector arranged in the fluid phase in the equilibrator, and that gases from the gas phase in the equilibrator is sucked into the ejector via a pipeline.

In one embodiment, an anti-foaming agent is arranged in the equilibrator.

In one embodiment, the anti-foaming agent is arranged in the equilibrator so that there is a gas phase above the anti-foaming agent.

In one embodiment, gases are sucked into the sensor device from the gas phase below or above the anti-foaming agent.

In one embodiment, gases returning from the sensor device are returned to the equilibrator via the gas phase (80a) above or below the anti-foaming agent.

In one embodiment, fluids are added to the equilibrator via a nozzle, arranged to distribute the water over the cross section of the equilibrator.

In one embodiment, the gas transporter is a diffusor.

In one embodiment, gases from the gas phase are directed via a pump from the anti-foaming agent to the diffusor.

In one embodiment, the equilibrator is arranged substantially horizontally and that gases are circulated in a closed loop through the gas phase in the equilibrator using a pump or propel.

In one embodiment, the sensor device is connected to the closed circuit.

In one embodiment, the fluid is transferred to the equilibrator via nozzles, and directed to the end edge of the equilibrator where it flows out through pipeline arranged with a water lock.

In one embodiment, the measurements of amount of gas are calibrated with measurements of a gas mixture, such as air, with a known gas composition.

In one embodiment, the calibration takes place in a closed circuit equipped with valves, and that the calibration is performed automatically at given times.

In one embodiment, fluid supplied to the equilibrator is obtained from a first container.

In one embodiment, the system comprises means to measure the pH in the container before and after addition of pH adjusting agent.

In one embodiment, the system comprises means to measure pH in the fluid in a container before addition of pH adjusting agent, and in container after addition of pH adjusting agent.

Alternatively, the container for pH adjustment may comprise means to measure pH both before and after addition of pH adjusting agent.

In one embodiment, the system comprises means to measure the amount of pH adjusting agent added.

In one embodiment, the system comprises means to measure pH in fluid after addition of pH adjusting agent, and that information about pH in the fluid after addition of pH adjusting agent is used to adjust the amount of pH adjusting agent being added to the container.

In another aspect, the present invention relates to a method to determine the amount of gas dissolved in a fluid, where the fluid is continuously added to an equilibrator arranged to set an equilibrium between the gases in a gas phase and the gasses dissolved in a fluid phase in the equilibrator, and where a pH adjusting agent to adjust the pH is added to the fluid before it is transferred to the equilibrator so that the equilibrium between said gases dissolved in the fluid and its ions dissolved in the fluid shift so that more gas is dissolved in the fluid.

In one embodiment, one or more gasses are added to the fluid phase to set the equilibrium between the gas phase and the fluid phase in the equilibrator more rapidly.

In one embodiment, gases from the gas phase in a closed volume is brought in contact with the fluid phase, and that a sensor device measures the amount of one or several gases in the gas phase.

In one embodiment, a gas transporter causes circulation of gasses from the gas phase to the fluid phase.

In one embodiment, the gas transporter is a pump and a pipeline for transport of gases from the gas phase to the fluid phase in the equilibrator.

In one embodiment, gasses are transported from the gas phase by a gas transporter to the fluid phase in a closed loop, and that a sensor device is arranged in the loop and measures the amount of one or more gasses in the gas phase.

In one embodiment, the gas from the gas phase is directed in a closed loop via a sensor device to measure the amount of a specific gas.

In one embodiment, the gas transporter is a hose equipped with an air pump to collect gas from the gas phase and add it to the fluid phase.

In one embodiment, the gas transporter is an ejector.

In one embodiment, the gas transporter is a diffusor.

In one embodiment, the sensor device measures the amount of one or more gasses selected from among hydrogen sulphide, carbon dioxide, oxygen, and ammonium.

In one embodiment, the said gas being measured is ammonium (NH₃)

In one embodiment, the flow-through velocity, and the amount of fluid through the equilibrator are measured or estimated, so that absolute amount of gas dissolved in the fluid may be calculated.

In one embodiment, the gas transporter generates micro-bubbles in the fluid phase.

In one embodiment, the fluid is transferred continuously from a first container to the equilibrator.

In one embodiment, a system is arranged according to the present invention, as indicated above, in several places in a fish farm.

In one embodiment, the system is arranged to measure amounts of gases in a fluid which is released into the farming tank.

In one embodiment, the system is arranged to measure amounts of gas released from the installation via the CO2 stripper.

In one embodiment, the system is arranged between one or more, or all of the modules in a fish farm, such as a RAS installation.

In one embodiment, the measurements are taken in real-time, and that a transmitter device on the sensor device transmits data to a controller unit.

In one embodiment, the system is set up with valves so that via programmable intervals it is possible to connect one calibrator gas with known concentrations to control drifting of the sensors.

In one embodiment, change of pH as a result of addition of pH adjusting agent is used to calculate the amount of fluid in a container based on measurements of amounts of said gas in the gas phase corrected for the change in amount of gas as a result of the change of pH.

In one embodiment, the amount of pH adjusting agent added is measured, and that amount of pH adjusting agent added is used to calculate the change of pH before and after addition of pH adjusting agent, and that change of pH is used to calculate amount of gas dissolved in fluid in container based on measurements of amounts of said gas in the has phase (80a) corrected for the change of amount of gas as a result of the change of pH.

### DESCRIPTION OF FIGURES

Below, preferred embodiments of the invention will be discussed in more detail with reference to the attached Figures, where:

FIG. 1 depicts a schematic of a system for measuring amount of a gas dissolved in a fluid. The fluid is transferred in a continuous flow to an equilibrator via a container for adjustment of the pH of the fluid.

FIG. 2 depicts the same solution as in FIG. 1, but in addition there is a gas transporter for transport of gases from the gas phase in the equilibrator to the fluid phase in the equilibrator.

FIG. 3 schematically depicts a solution where the gas transporter is an ejector.

FIG. 4 schematically depicts a solution where the gas transporter is a diffuser.

FIG. 5 schematically depicts a system where the equilibrator is arranged horizontally.

FIG. 6 depicts a system where systems for measurement of gases may be used in an arrangement at an RAS installation.

FIG. 7 depicts the same embodiment as FIG. 1, but where a container/chute for addition of pH adjusting agent to the container 20 is depicted.

### DETAILED DESCRIPTION OF PREFERRED EMBODIMENTS OF THE INVENTION

As mentioned above, there are no solutions available to measure the low concentrations of dissolved NH₃ in fluid that are formed in fish farms. Even low levels of NH₃ is harmful to the fish, and with the present invention a solution is provided which makes it possible to detect extremely low levels of NH₃, i.e., levels of NH₃ in the range harmful for the fish.

In fluids, the amount of NH₃ gas dissolved in the fluid is an equilibrium with ammonium ions NH⁴⁺. In a normal fish farm, this equilibrium of NH₃ in fluid is influenced by the pH of the fluid. The pH of the fluid in a normal fish farm, including a RAS installation, is in the ranges of 6.8 to 7.5. As is shown in the figure below, a change in pH to a more alkaline value will displace the equilibrium between NH₃(aq) and NH⁴+(aq) and settle at a level where the relationship between NH₃ and NH⁴⁺ increases, i.e., more NH₃ gas will be dissolved in the fluid. Thereby, it is possible to increase the amount of NH₃ gas dissolved in fluid to a level which it is practically possible to measure. Other conditions influencing the equilibrium between NH₃ and NH⁴⁺, is temperature and saline content of the fluid. Therefore, it is necessary to use a table, and perform the corrections which apply to the actual temperature and the actual saline content.

It is not possible to perform this displacement of the equilibrium, i.e., increase pH, in the farming installation itself as the amounts of NH₃ that will form will be highly toxic to the fish in the installation.

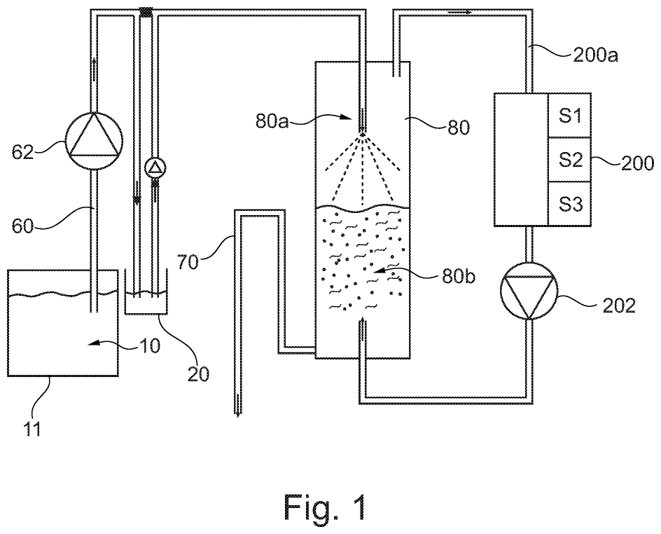

As shown in FIG. 1, fluid is transferred from the farming installation, depicted as container 11 in FIG. 1, via a container 20 to adjust pH of the fluid, before the fluid is directed to an equilibrator 80. A pH adjusting agent is added to the fluid in the pH adjusting container 20, so that the pH of the fluid transferred to the equilibrator 80 changes, so that the equilibrium between gas and ions dissolved in the fluid changes. By adding an alkaline solution, such as for example NaOH, to a fluid containing NH₃, the pH of the fluid downstream of the container 20 will increase, and this shift in the equilibrium forms more NH₃ in the solution. Thereafter, the pH adjusted fluid is transferred to an equilibrator 80 where an equilibrium is set between NH₃ in the fluid and NH₃ in the gas phase 80a over the fluid 80b. Sensors 200 measure the amount of NH₃ in the gas phase 80a over the fluid 80b. Based on the increase of pH (as either measured or estimated) for the fluid in container 20, and which is added to the equilibrator 80, it is possible to use established tables (as outlined schematically in the figure above) and estimate the increase of the proportionality of NH₃:NH⁴⁺, and thus calculate the amount of NH₃ originally dissolved in the container 11 (i.e., before adjusting the pH).

Thus, the core of the invention is to transfer the fluid to the equilibrator 80 so that the amount of gas may be measured in the gas phase 80a that sets above the fluid phase 80b in the equilibrator 80, and that in addition, the pH of the fluid is adjusted before it is fed into the equilibrator 80 to influence the equilibrium between gas dissolved in fluid and the corresponding ions in the fluid. This is explained with reference to the figure above for the NH₃—NH⁴⁺ equilibrium which is influenced by the pH of the solution. A higher, more alkaline pH displaces the equilibrium so that the fluid contains proportionally more NH₃. This brings the amount of NH₃ up to levels measurable in the gas phase 80a in an equilibrator 80. By calculating added amount of pH adjusting agent (such as NaOH) or by measuring the pH before and after addition of pH adjusting agent, it is possible to calculate how many times the NH₃ concentration has increased in the fluid, and it is possible from the measurements of NH₃ after pH adjustment to calculate how much NH₃ the original fluid of the farming installation contained. Thereby, a system and method is provided to measure amounts of NH₃ even when they are so low that they cannot be measured with conventional measuring methods. In many cases it will not be necessary to measure absolute amounts of NH₃, as it will be sufficient to consider the development of NH₃ over time. The method according to the invention is performed continuously and monitors the relative measurement of NH₃. It is also possible to carry out chemical measurements of amount of NH₃ in the farming container 11 and relate these to the measured values of NH₃ in the pH adjusted fluid in the equilibrator 80.

In PCT application PCT/NO2020/050280 the proprietor of the present patent application has described transfer of fluid to an equilibrator to allow for the measurement of low amounts of gasses dissolved in a fluid. The proprietor is active in the aqua culture industry, and the invention of PCT/NO2020/050280 is exemplified by measurements of hydrogen sulphide gas, i.e., H₂S (aq) in a fluid. The present invention relates to an improved measuring method for gases in fluid, for gases where an adjustment of pH increases the amount of gas in the fluid by displacing the equilibrium between gas and ions in the fluid more towards gas, either by increasing the pH of the fluid (as with the NH₃ system) or by reducing the pH.

FIG. 1 schematically depicts such a system for adjusting the pH in a fluid before the amount of gas in the fluid is measured in an equilibrator 80. It is desirable to measure the amount or concentration of a given gas in a fluid contained in a container 11. For example, this may be a fish farm, such as a RAS installation. The gas it is desirable to measure, may for example be NH₃, but the method may also be utilized for other gases where a change of pH will change the equilibrium between the gas and its ions in solution. Conventional methods to measure the amount of gas for measurement of many types of gases, such as NH₃, are not sufficiently sensitive to enable measurement of the amount of gas in the fluid of the container 11 itself. Therefore, fluid is transferred to an equilibrator 80 via pipelines 60, using a pump 62.

Downstream of the container 11 (for example the farming tank in the fish farm), the fluid is transferred to a container 20 for adjustment of the pH. In case of measurement of NH₃, an alkaline will be added to the container 20, i.e., an agent adjusting the pH to higher, more alkaline values. An example of such an agent, is NaOH. The pH adjusted fluid is then fed from container 20 to equilibrator 80.

In association with the equilibrator 80, a water lock 70 is arranged at the outlet to enable the regulation of the level of fluid in the equilibrator 80. After a given time, an equilibrium will set for the gas it is desirable to measure, between amount of gas dissolved in the fluid 80b in the equilibrator 80 and amount of gas dissolved in the gas phase 80a above the fluid level in the equilibrator 80. It is preferable that this equilibrium between gas dissolved in the fluid phase 80b and the gas phase 80a, respectively, sets rapidly so that it is possible to continuously carry out the measurements of actual amounts of the gas, which is measured using sensors 200 in the gas phase 80a. To effectuate a rapid setting of this equilibrium between gas in the fluid phase 80b and the gas phase 80a, the system is preferably equipped with means to cause a circulation of the gas phase 80a to the fluid phase 80b. If the gases from the gas phase 80a are transported to the fluid phase 80b, and preferably also transported through the fluid phase 80b, then the equilibrium between gases in the fluid phase 80b and the gas phase 80a will set more rapidly. These means to transport gases through the fluid phase 80b are in some of the figures schematically depicted as a gas transporter with reference number 100. In a simple, preferred embodiment the gas measured in sensor 200 is transported to a lower level in the fluid phase 80b so that bubbles of gas phase 80a raise up through the fluid phase 80b.

It is not necessary to use gases from the gas phase 80a for transport of gases through the fluid phase 80b. Any gas directed through the fluid phase 80b will cause a more rapid setting of the equilibrium between gas in the gas phase 80a and the fluid phase 80b. Therefore, it is often preferable to bubble another gas, such as air or oxygen, through the fluid phase 80b to cause this more rapid setting of the fluid phase. For example, it is possible to add air or oxygen using an injector or ejector directly into the fluid phase 80b. It is preferred that the gas (for example air) which is to be added to the fluid phase 80b, form small bubbles, preferably micro-bubbles, in the fluid phase 80b. Such bubbles, and preferably micro-bubbles, establishes a rather large interfacing surface between gases in the gas phase 80a (which also comprise the volume inside the bubbles) and gases in the fluid phase 80b. A larger interfacing surface accelerates the establishment of the equilibrium.

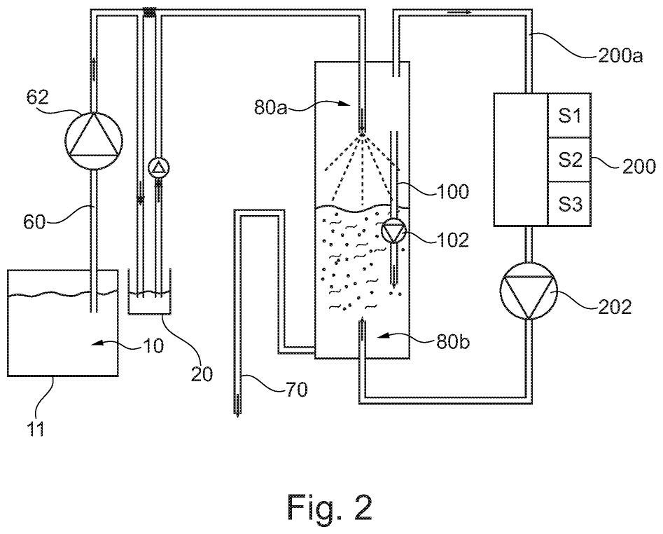

Addition of gas or gasses to the fluid phase 80b may be carried out in many ways, and the gas transporters may therefore be different. In FIG. 2, this gas transporter 100 is schematically depicted arranged inside the equilibrator 80, but in an alternative embodiment it has been arranged on the outside of the equilibrator 80 but where the pipelines stretch through the equilibrator 80 so that gasses may be transferred from 80a to 80b, i.e. gases are extracted from the gas phase 80a and added, preferably at a lower level, to the fluid phase 80b. Trials have demonstrated that it is favourable that the gases released from the gas transporter in the fluid phase 80b are in the form of small air bubbles, preferably as micro-bubbles. As mentioned above, micro-bubbles have a large surface compared to volume, i.e. a relatively large interface surface between fluid and gas, and this causes an efficient exchange of gases between 80a and 80b, and a rapid setting of the equilibrium in the equilibrator 80. In FIG. 2, the supply of pH adjusting agent is schematically depicted by the fluid from container 11 being led through container 20.

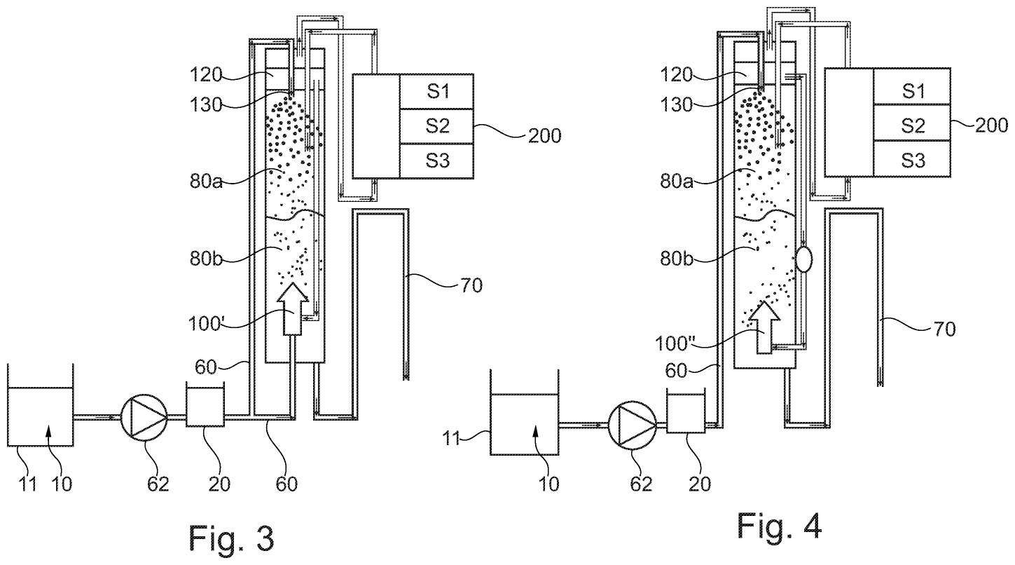

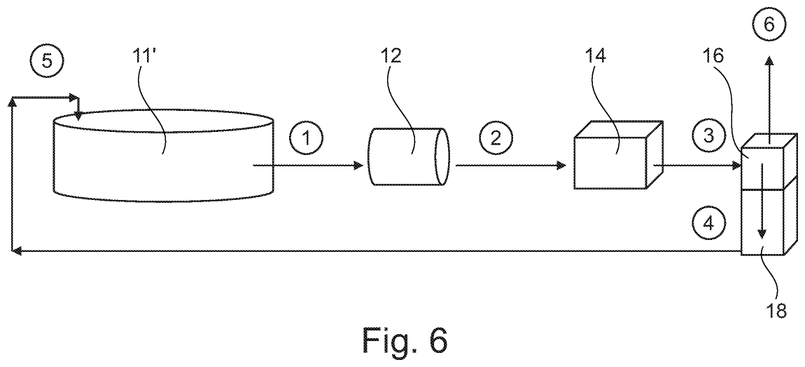

FIG. 3 (FIG. 6), an embodiment of the invention where an ejector 100′ arranged in the equilibrator 80 is used to generate air bubbles in the fluid phase 80b. Fluid 10 from container 11 is fed via pump 62 and pipelines 60 to both the top of the equilibrator 80 and to an ejector 100′ arranged in the fluid phase 80b in the equilibrator. Gases from the gas phase 80a are sucked into the ejector 100′ via pipeline 100. FIG. 3 also depicts some other elements which improve the system and the method. Using ejector 100′, some foam is generated depending on type of fluid 10. Therefore, FIG. 3 depicts an anti-foaming agent 120 arranged in the equilibrator 80, which reduces the amount of foam in the gas phase 80a. It is further preferred that the fluid 10 from container 11 is directed via this anti-foaming agent 120 to the equilibrator 80.

The anti-foaming agent 120 may be placed at different levels of the equilibrator 80 Above the anti-foaming agent 120 there is a gas space, where for example it is possible to suction gases to the sensor box 200. Foam should not enter into this space. Gases returning from the sensor box 200 travel through the anti-foaming agent 120 so that these gases interchange with gases arriving from the ejector 100′. Should foam enter the anti-foaming agent 120, it will be sucked down again to the ejector 100′ together with the gases. When foam is sucked down to the ejector 100′, then the function of this will be impaired and therefore also generate less foam. In this manner, we prevent foam for crossing over to the anti-foaming agent 120. The anti-foaming agent 120 has openings 120a causing the gases to circulate through it, but foam with higher density is sucked into the return and down to the ejector 100′. FIG. 3 also shows that fluid 10 arriving from container 20 is distributed via a nozzle 130. This nozzle 130 distributes the water across the entire cross section of the equilibrator 80 and provides a good gas exchange between the gas phase 80a and the fluid phase 80b. During experiments with this embodiment, it has been shown that this nozzle provides such an efficient gas exchange that it is not necessary to utilize an ejector or diffuser, i.e., the solution with nozzle 130 is utilized together with the embodiments of gas transporter 100 shown in FIGS. 1 and 2.

FIG. 4 depicts a similar embodiment, but where the ejector 100′ has been replaced by a diffuser 100″ (fizz-rock) which directs gases from the gas phase 80a through a pump 102″ from the anti-foaming agent 120 to a diffuser 100″ which is arranged in the fluid phase 80b. This solution with diffuser 100″ may also be implemented without anti-foaming agent 120 and nozzle 130, but these solutions are not shown in FIG. 4.

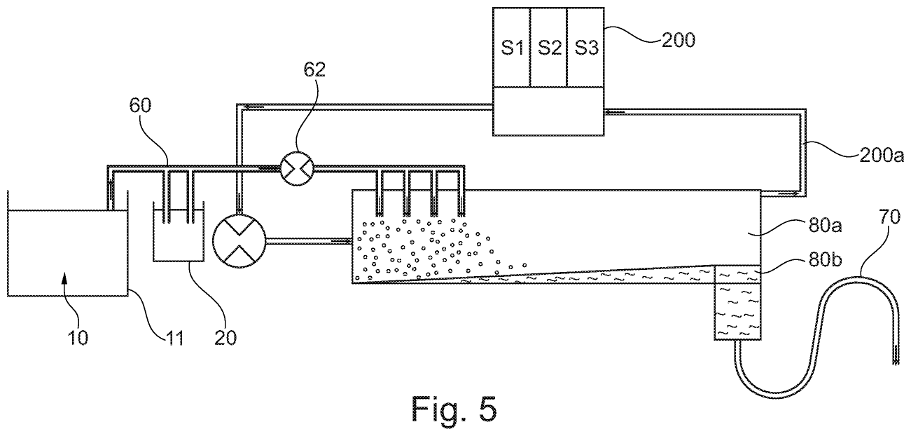

FIG. 5 depicts a solution where the equilibrator 80 is arranged horizontally and gases circulated in a closed circuit through the gas phase 80a in the equilibrator 80 by use of a pump or a propel. The sensor arrangement 200 may also be connected to this closed circuit. The fluid 10 is transferred from container 11 via container 20 and dropped through shower heads 130′ and directed to the end edge of the equilibrator 80 where it runs through pipeline 70 with a water lock regulating the height of the water level in the equilibrator 80.

FIG. 6 depicts an embodiment where the system and method according to the invention is used several places in a typical RAS farming installation. The Figure schematically depicts how fluid from the farming tank 11′ is transferred to a drum filter 12, thereafter to a biofilter 14 and then to a CO2 ventilator 16/18, and back to the farming tank 11′. In the transfer between each of these units, and from the CO2 stripper also, where gases leave the system, it is possible to use a system and a method according to the present invention to measure the concentration of gases present in the fluid in the equilibrator 80 and calculate the amount of these gasses originally present in the container 11. In an aqua culture installation, it is first and foremost relevant to measure the concentration of the gases H₂S, CO2, NH₃, and O₂. The system according to the invention may thus measure the amounts of gases in the fluid that is directed into the installation, shown with reference number 5 in FIG. 6. At position 1, the level of gases in the fluid leaving the farming tank 11′, and changes in level between positions 1 and 5, indicate the change of amounts of gas that have taken place in the farming container 11′. Furthermore, the system according to the invention may be arranged between different components in the RAS installation, such as indicated at positions 2, 3, and 4. The system at position 6 can measure amounts of gas released from the RAS installation. In this way, it is therefore possible for example to identify whether a biofilter has accumulated too much organic material so that it starts to produce H₂S. Should the level of H₂S raise the farmer may implement necessary remedies. Correspondingly, it is necessary to implement remedies when amounts of NH₃ harmful to the fish are measured.

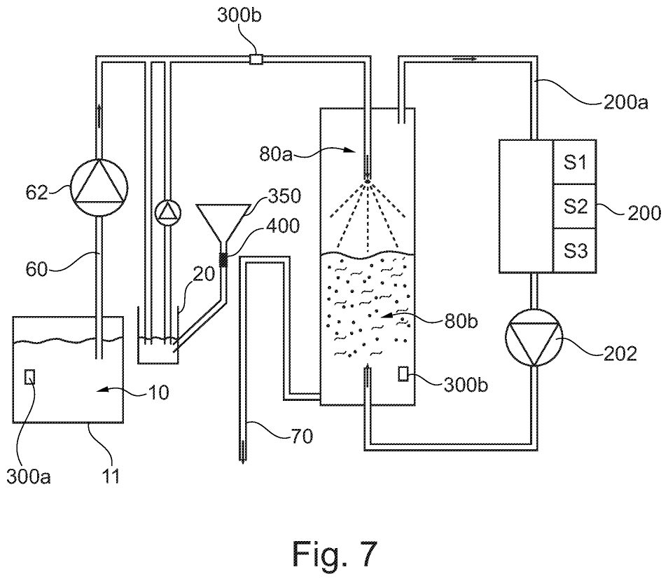

FIG. 7 schematically depicts an embodiment where sensors 300 are arranged in the system to measure pH in the system. In an embodiment described above, sensors 300a are arranged to measure the pH of the farming container 11 itself, so that it is possible at any time to measure pH before adding pH adjusting agent as the fluid is directed via container 20. Furthermore, it is possible to measure pH using sensor 300b to measure pH in the fluid phase 80b of the solution present in the equilibrator 80. The sensor 300b to measure pH after addition of pH adjusting agent, may also be arranged in the pipeline leading the fluid 10 from the container 20 for adjustment of pH to the equilibrator 80. Conventional pH gauges may be used, and these are preferable read off automatically, and the results sent to a controller unit so that it is possible to at any time have the overview over pH in the solution before addition of pH adjusting agent, and also what the pH will be after addition. Based on this change of pH, and the mentioned tables and diagrams, it is then possible to calculate how many times the concentration of NH₃ has changed since addition of pH adjusting agent, and it is possible to correct back and thereby calculate how much NH₃ was present originally in the fluid 10, i.e., before addition of pH adjusting agent. By adjusting the pH before measuring NH₃, the sensitivity of the measurement is increased as the equilibrium between gas dissolved in fluid, and its corresponding ions dissolved in fluid to a pH value where the equilibrium is displaced towards the gas. For the NH₃ system, this means that if the pH of the fluid is increased, then the amount of NH₃ (aq) in the fluid 10 increases.

Alternatively, measuring pH both before and after addition of pH adjusting agent, it is possible to dosage in pH adjusting agent to obtain complete control over how much agent is added. When it is known how much pH adjusting agent has been added, and what also effect this addition has on the equilibrium of the fluid 10, then it is possible to calculate what the pH value will be after addition and use this calculated value to determine how many times amount of the relevant gas, such as NH₃, have increased with the pH adjustment. In FIG. 7, a system is schematically depicted where a container 350 is arranged for the addition of pH adjusting agent. The pipeline leading from container 350 to container 20 is equipped with a dosing unit 400 controlling and measuring the amount of pH adjusting agent being added. If the pH adjusting agent is a liquid, then it is preferred that the dosing unit measures the amount of volume added, for example the amount of millilitres pH adjusting liquid added. If the pH adjusting agent is a powder, then the dosing unit 400 may preferably administrate and measure dose based on weight. Such dosing units 400 are conventional and may be bought.

The system and method according to the invention is described for measurement of NH₃ in a farming installation, but we would like to emphasise that also other gasses may be measured, and then in particular other gasses shifting the equilibrium between gas and ions dissolved in fluid if a change of pH is enforced. We also want to emphasise that other gases may be measured using the system and the method, i.e., without adjustment of pH, or without this influencing the amount of said gas in the fluid 10, that it is possible to actually use the effect of transferring the fluid to an equilibrator to enable the measurement of amount of gas in the gas phase 80a and not in the fluid 10 itself. In addition, we want to emphasise that it is possible to measure several gases at the same time using several sensors 200, where each one of them is specific for at least one of the mentioned gases to be measured. With the invention is provided an option for continuous measurement of gases in fluids in an installation, such as a farming installation. Separately, and by combining the two principles; (i) measurement of gas level in the gas phase when the fluid is in an equilibrator 80 and is set to an equilibrium between said gas in the gas phase 80a and the fluid phase 80b, and ii) change of pH in the fluid 10 to displace the equilibrium towards more dissolved gas in the fluid to be able to indirectly measure smaller amounts of gas in fluid, are provided new means to maintain continuous control over the concentration of gases in fluid, and especially gases that may have harmful effects on the species, such as fish, being farmed in the fluid 10 in container 11. With the method and the system is provided a possibility for obtaining continuous control over the development of gases in the fluid. Measuring of relative values is simple, i.e., measuring changes in amount of a given gas, but it is also simple to calculate the absolute values and clarify whether these are approaching a level that will be harmful for the fish so that remedies must be implemented.

Below follows a more detailed description of how the method according to the invention is performed for measurement of NH₃. The embodiment may be as depicted in FIG. 1.

The challenges associated with measuring NH₃ at low ppb levels, is that existing sensors do not have a sufficiently accurate (sufficiently sensitive) measuring range. Therefore, we will adjust up pH as mentioned previously, to increase the percentage of NH₃, so that we obtain an amount of dissolved NH₃ within a measurable level.

The signal emitted from a typical NH₃ sensor is in the form of an analogue tension in the dimension of about 15 μV per ppb. To measure these low levels, it is necessary to build a conditioning circuit which adapts the signal emitted from the sensor to a sensible measuring range for an A/D converter. In this embodiment, we use a 16 bits A/D converter with a measuring range of 0 to 3.3 Volt. To optimally utilize the measuring range of the converter, the conditioning circuit must remove DC offset from the sensor and amplify the signal at the same time so that it fits the entry stage of the A/D converter. In this embodiment, precision tension reference and differential amplifiers are used to convert the signal emitted from the sensor to suitable values for the A/D converter. To reduce the noise, both analogue and digital noise filters are implemented. Digital filtering is necessary to smooth out the signal. This filter may have a time constant of typically around 5 mins.

The signal emitted from the conditioning circuit is sent to the A/D converter, which converts mV voltage to a 16 bits number. We now have a scale where 1 ppb concentration is equivalent to approximately 2 stages on the A/D converter. We have managed to obtain a mV voltage which depends on the NH₃ level of the gas we are measuring, and that the mV signal is in a detectable range for A/D conversion.

However, this mV voltage strongly depends on varying temperature, and to enable conversion from mV to NH₃ concentration, it is first necessary to determine the relationship between temperature and mV at a given concentration of NH₃.

This is carried out experimentally by obtaining a long series of measurements where the sensor first has clean air (NH₃-0) and then reading mV voltage from the sensor at varying temperatures. Thereafter, we build up a table over mV voltage vs. temperature, where temperature ranges from for example 0 to 20° C. in 1° ° C. intervals.

When this is done, the same experiment is made over again, but now with sensor exposed to air with an upper limit of NH₃ concentration. The air is in a closed circuit, where we establish equilibrium between gas and fluid phase as described. Using established set of formula, we calculate the NH₃ concentration in the water being measured.

The tables we build in this manner, are converted to liners or polynomic trend lines which are then used in the set of formulas to implement temperature correction.

The sensor is linear in the range of interest to us. Using the performed tests, we have arrived at mV distension at the lower and higher measuring ranges. These values may be used to define the formula to convert from mV to temperature corrected ppb NH₃. The sensor will measure the NH₃ gas concentration at intervals of 1 second.

### Example 1

A practical test was performed where a mixture of with a concentration of Total ammonium of approximately 2 mg/L.

As indicated above, the proportion of NH₃ in water with a certain Total Ammonium Level depends mainly on pH, and less on temperature and salinity.

Ammonium chloride in a concentration of 9% with NH⁴OH was used. Approximately 0.5 ml ammonium chloride was added to a bucket 10L bucket. pH was measured to 7.5. Approximately 1 litre of the water was added to a container with a lid. A small air pocket was left at the top of the container. The container was shaken so that the gas in the air pocket was brought into equilibrium with the water. The gas phase above the water and the amount of gas in the water set as an equilibrium, similar to the equilibrator explained above. Thereafter, the NH₃ sensor was placed under the lid and the concentration of NH₃ in the gas phase under the lid was measured. It was measure to 0.015 mg/L (10 ppb in air) using a gas sensor of the type Aquasense. This is estimated to approximately 0.9% of Total Ammonium in the container. An alkaline was then added to the container to raise the pH. pH was measured to approximately 9.0. Thereafter, the lid was put back on and the container shaken for air to be brought into equilibrium with the water. The concentration of NH₃ was measured once again and now showed 0.40 mg/L (approximately 270 ppb in air) using the same sensor of the type Aquasense. By calculations, the proportion of NH₃ should be approximately 21.5% of Total Ammonium.

This demonstrates that by raising the pH in the water to be measured for amount of NH₃ gas dissolved, the equilibrium will be displaced towards NH₃ gas (as explained above) and the fraction of NH₃ will increase considerably (more than 20 times). This means that it is possible to indirectly measure amounts of NH₃ that are more than 20 times lower than when the pH is not adjusted, and it is therefore possible to utilise sensors with a range that is higher and more easily accessible thereby.

By use of known formulas/tables it is thereafter possible to calculate back to the level that was in the water before an alkaline was added to increase the pH.

1.-55. (canceled)

56. A system to determine the amount of gas dissolved in a fluid (10) in a container (11), characterized by the system comprising an equilibrator (80) arranged to set an equilibrium between gases in a gas phase (80a) and a fluid phase (80b), a sensor device (200) for measurement of amount of gas in the gas phase (80a), and a container (20) upstream of the equilibrator (80) and downstream of the container (11) for regulation of pH in the fluid (10) before it is transferred to the equilibrator (80).

57. The system according to claim 56, characterized by that the container (20) arranged to regulate pH of the fluid (10) comprised means (20a) to add a pH adjusting agent to the container (20).

58. The system according to claim 56, characterized by that the system comprises a gas transporter (100, 100′, 100″) arranged to cause circulation of gases from the gas phase (80a) to the fluid phase (80b).

59. The system according to claim 56, characterized by that the equilibrator has an outlet (70) with a water lock to regulate the water level in the equilibrator (80).

60. The system according to claim 56, characterized by that one or more gasses are added to the fluid phase 80b.

61. The system according to claim 58, characterized by that the gas transporter (100, 100′, 100″) transports gasses in a closed circuit from the gas phase (80a) to the fluid phase (80b).

62. The system according to claim 58, characterized by that the gas transporter (100, 100′, 100″) comprises a pump (102) and a pipeline (100) for transport of gasses from the gas phase (80a) to the fluid phase (80b).

63. The system according to claim 56, characterized by that gas from the gas phase (80a) is directed in a closed loop via a sensor device (200) to measure the amount of a given gas.

64. The system according to claim 56, characterized by that in the equilibrator (80) a pump with an anti-foaming agent (120) is arranged in the gas phase (80a).

65. The system according to claim 56, characterized by that the equilibrator (80) is arranged substantially horizontally and that gases are circulated in a closed circuit through the gas phase (80a) in the equilibrator (80) using a pump or propel.

66. The system according to claim 56, characterized by the that the calibration takes place in a closed circuit equipped with valves, and that the calibration is carried out automatically at given points in time.

67. The system according to claim 56, characterized by that fluid (10) supplied to the equilibrator (80) is sourced from a first container (11).

68. A method to determine the amount of gas dissolved in a fluid (10), wherein the fluid (10) is continuously supplied to an equilibrator (80) and directed at setting an equilibrium between the gasses in a gas phase (80a) and the gasses dissolved in a fluid phase (80b) in the equilibrator (80), and wherein a pH adjusting agent is added to the fluid (10) after having left the container (11) and before it is transferred to the equilibrator (80) to adjust the pH of the fluid (10) so that the equilibrium between said gas dissolved in the fluid 10 and its ions dissolved in the fluid (10) shifts, so that more gas is dissolved in the fluid (10).

69. The method according to claim 68, characterized by that one or several gasses are added to the fluid phase (80b) to more rapidly set the equilibrium between the gas phase (80a) and the fluid phase (80b).

70. The method according to claim 69, characterized by that gasses from the gas phase (80a) in a closed gas volume are brought into contact with the fluid phase (80b), and that a sensor devise (200) measures amount of one or more gasses in the gas phase (80a).

71. The method according to claim 68, characterized by a gas transporter (100, 100′, 100″) causes circulation of gasses from the gas phase (80a) to the fluid phase (80b).

72. The method according to claim 70, characterized by that the said gas is ammonium (NH₃).

73. The method according to claim 68, characterized by that the flow through velocity and amount of fluid through the equilibrator is measured or estimated, so that absolute amount of gas dissolved in the fluid (10) may be calculated.

74. The method according to claim 71, characterized by that the gas transporter (100, 100′, 100″) generates micro-bubbles to the fluid phase (80b).

75. The method according to claim 68, characterized by that the fluid (10) is transferred continuously from a first container (11) to the equilibrator (80).
